(19) United States
(12) Patent Application Publication
(10) Pub. No.: US 2020/0071290 A1
(43) Pub. Date: Mar. 5, 2020

(54) Pyrazole Amine Reactive Crystallization

(71) Applicant: Dow AgroSciences LLC, Indianapolis, IN (US)

(72) Inventors: Deboleen Chakraborty, Midland, MI (US); Xiaoyong Li, Midland, MI (US); Todd William Toyzan, Freeland, MI (US); Ronald B Leng, Midland, MI (US)

(73) Assignee: Dow AgroSciences LLC, Indianapolis, IN (US)

(21) Appl. No.: 16/611,636

(22) PCT Filed: May 24, 2018
(86) PCT No.: PCT/US2018/034283

§ 371 Date: Nov. 7, 2019

Related U.S. Application Data
(60) Provisional application No. 62/511,391, filed on May 26, 2017.

(51) Int. Cl. C07D 401/04

(52) U.S. Cl. CPC C07D 401/04

(57) ABSTRACT

This application relates to efficient and economical synthetic chemical processes for the preparation of pesticidal thioethers. Specifically, the present application relates to improved reactive crystallization methods for producing compounds useful in the preparation of pesticidal thioethers.

### CROSS-REFERENCE TO RELATED APPLICATIONS

This is a national phase entry under 35 U.S.C. § 371 of international patent PCT/US2018/034283, filed on May 24, 2018 and published in English as international patent publication WO2018/217966 on Nov. 29, 2018, which claims priority to U.S. Provisional Application No. 62/511,391, filed May 26, 2017, which is incorporated herein by this reference in its entirety.

### TECHNICAL FIELD

This application relates to efficient and economical synthetic chemical processes for the preparation of pesticidal thioethers. Specifically, the present application relates to improved reactive crystallization methods for producing compounds useful in the preparation of pesticidal thioethers.

### BACKGROUND

There are more than ten thousand species of pests that cause losses in agriculture. The worldwide agricultural losses amount to billions of U.S. dollars each year. Stored food pests eat and adulterate stored food. The worldwide stored food losses amount to billions of U.S. dollars each year, hut more importantly, deprive people of needed food. Certain pests have developed resistance to pesticides in current use. Hundreds of pest species are resistant to one or more pesticides. The development of resistance to some of the older pesticides, such as DDT, the carbamates, and the organophosphates, is well known. However, resistance has even developed to some of the newer pesticides. As a result, there is an acute need for new pesticides that has led to the development of new pesticides. Specifically. US 20130288893(A1) describes, inter alia, certain pesticidal thioethers and their use as pesticides. Such compounds are finding use in agriculture for the control of pests.

Because there is a need for very large quantities of pesticides, specifically pesticidal thioethers, it would be advantageous to produce pesticidal thioethers efficiently and in high yield from commercially available starting materials to provide the market with more economical sources of much needed pesticides.

### DEFINITIONS

As used herein, the term “alkyl” includes a chain of carbon atoms, which is optionally branched including but not limited to C₁-C₆, C₁-C₄, and C₁-C₃. Illustrative alkyl groups include, but are not limited to, methyl, ethyl, n-propyl, isopropyl, n-butyl, isobutyl, sec-butyl, tent-butyl, pentyl, 2-pentyl, 3-pentyl, and the like. Alkyl may be substituted or unsubstituted. As used herein, the term “alkynyl” includes a chain of carbon atoms, which is optionally branched, including but not limited to C₁-C₆, C₁-C₄, and C₁-C₃, and has at least one carbon-carbon triple bond (CC). Illustrative alkynyl groups include, but are not limited to, 1-propyn-1-yl, 1-propyn-3-yl, 1-butyn-3-yl, 1-butyn-1-yl, 2-butyn-1-yl, 1-pentyn-1yl, 2-p entyn-1-yl, 3-pentyn-1-yl, and the like. Alkynyl may be substituted or unsubstituted.

### DETAILED DESCRIPTION

Processes for the preparation of the compound of the formula

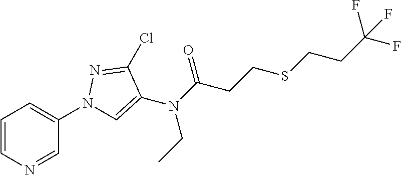 [SMILES: CCN(C(=O)CCSCCC(F)(F)F)c1cn(-c2cccnc2)nc1Cl]

are described in, for example, US 20130288893(A1) and in U.S. Pat. No. 9,102,655. One such process involves the preparation of 3-Chloro-N-ethyl-1-(pyridin-3-yl)-1H-pyrazol-amine (1d) according to scheme (1).

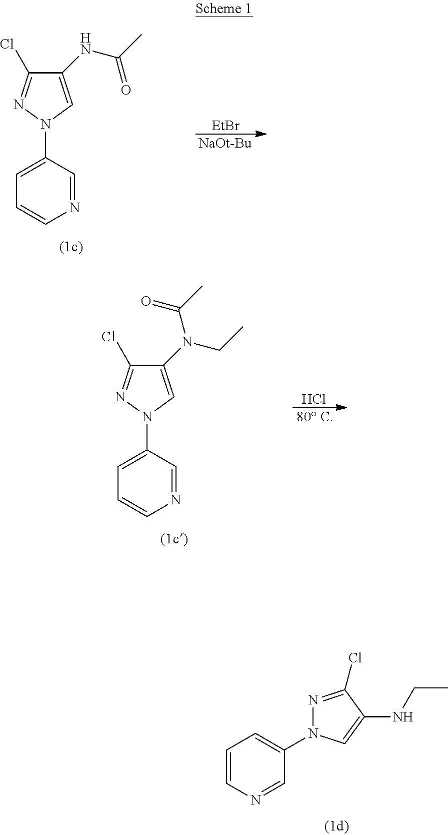

The process described in Scheme 1 involves the alkylation of intermediate compound (1c) to form compound (1c′), which is subsequently hydrolyzed to intermediate compound (1d).

The process, as exemplified in U.S. Pat. No. 9,102,655 involves the purification of (1c′) by semi-automated silica gel chromatography. While effective for producing purified intermediate compound (1c′) on a laboratory scale, such a purification step is inefficient and expensive when the process is scaled up, especially for commercial scale production. As a result, for larger scale applications, the intermediate compound (1c′) is carried through the hydrolysis step to the formation of (1d) without purification.

In some embodiments, the product of the hydrolysis reaction when intermediate compound (1c′) is treated with a strong acid, such as HCl, is the diacid salt (1d′) which is neutralized to form the desired product (1d) as shown in Scheme 2.

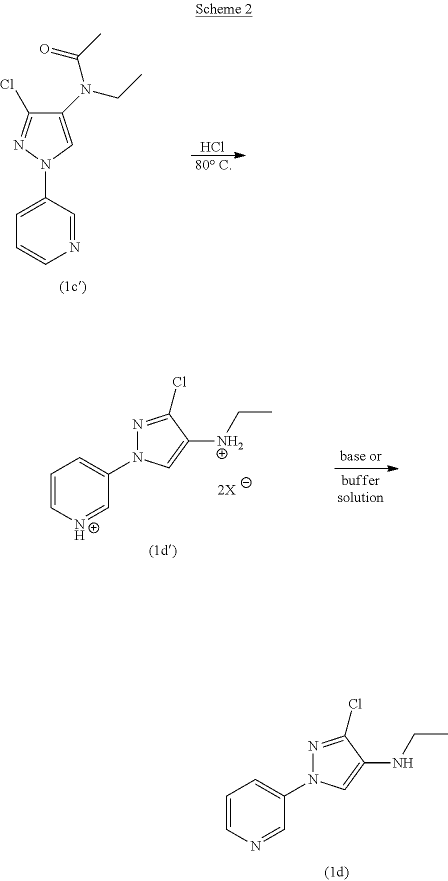

The product isolation step in the conversion of intermediate compound (1c′) into intermediate compound (1d) involves a pH swing reactive crystallization by the addition of an aqueous base solution (25-50% NaOH solution, pH ˜14) to the highly acidic (pH ˜0) crude aqueous hydrolysis reaction mixture to convert intermediate compound (1d′) into the pyrazole amine intermediate compound (1d). The addition of aqueous base to the crude hydrolysis reaction mixture results in the crystallization of the pyrazole amine intermediate compound (1d). During the course of the base addition, the pH of the hydrolysis reaction mixture changes from about 0 to the desired end-point of about 8-10.

It was discovered that the pyrazole amine compound (1d) has the propensity to oil at around pH 2.7 and at pH >12.5. Intense oiling and rinding during the pH swing crystallization (adding base to the acid) lead to processability issues, low product purity (<88%) and low isolated product yield (<85%). Propensity to oiling during reactive crystallization was found to be positively dependent on the wt % of residual THF in the reaction mixture. Oiling at pH ˜2.7 was observed even for no residual THF indicating that oiling is an intrinsic property of the molecule. Without being bound by theory, it is believed that at pH ˜2.7, the molecule is partially neutralized to the mono acid addition salt, which may lead to oiling. Oiling at lower pH led to substantial processability issues leading to poor yield and compromised product purity.

It has been surprisingly discovered that the oiling problems associated with the reactive crystallization technique for purifying pyrazole amine intermediate compound (1d) can be overcome via a reverse addition process where the acidic aqueous hydrolysis reaction mixture is added into a basic solution (e.g. a mild aqueous base, and organic base or buffer system). It has been surprisingly discovered that the reverse addition reactive crystallization technique described herein completely avoided oiling issues during the reactive crystallization of pyrazole amine intermediate compound (1d) irrespective of the THF content of the reaction mixture. As a result of the inventive process, the processability, yield, and product purity of pyrazole amine intermediate compound (1d) were significantly improved.

It will be appreciated that the reactive crystallization technique described herein can be used for purifying and isolating any intermediate described in Scheme 2, as required, or in any similar process known in the art for preparing any of the intermediates shown in Scheme 2, or structural variants thereof. In some embodiments, the present disclosure provides a process for producing a compound of the formula (1d-1)

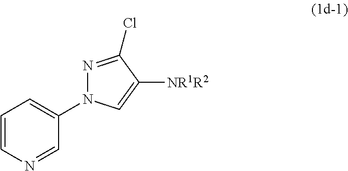

in purified form, wherein R¹ and R² are each independently H, C₁-C₄ alkyl, C₁-C₄ alkynyl, or —C(O)C₁-C₄ alkyl, comprising

a. contacting a compound of the formula (1d′-1)

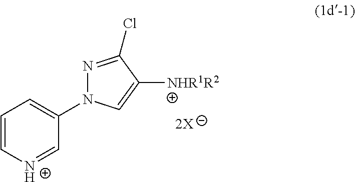

wherein R¹ and R² are each independently H, C₁-C₄ alkyl, C₁-C₄ alkynyl, or —C(O)C₁-C₄ alkyl, X⁻ is an anion, with a base or a buffer system at a pH of from about 7 to about 12 and at a temperature of from about 20° C. to about 35° C., to provide a suspension mixture of the compound of the formula (1d-1) as a solid product suspended in the base or the buffer system, and optionally also including the steps of

b. isolating the compound of the formula (1d-1) to provide the compound of the formula (1d-1) in purified form, and

c. drying the compound of the formula (1d-1) in purified form in vacuo.

In some embodiments, the present disclosure provides a process for producing a compound of the formula (1d)

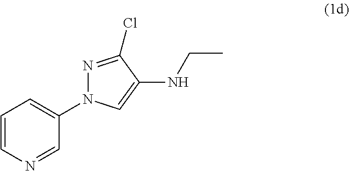

in purified form comprising

a. contacting a compound of the formula (1d′)

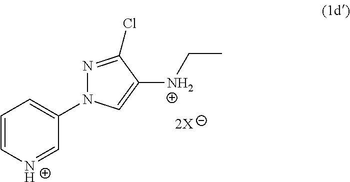

wherein X⁻ is an anion, with a base or a buffer system at a pH of from about 7 to about 12 and at a temperature of from about 20° C. to about 35° C., to provide a suspension mixture of the compound of the formula (1d) as a solid product suspended in the base or the buffer system, and optionally also including the steps of

b. isolating to provide the compound of the formula (1d) in purified form, and

c. drying the compound of the formula (1d) in purified form in vacuo.

In some embodiments, the step of contacting comprises adding an acidic aqueous mixture comprising the compound of the formula (1d′-1) or the compound of the formula (1d′) to the base or buffer system. In some embodiments, the acidic aqueous mixture further comprises at least one organic solvent. The organic solvent can be present in an amount of about 0.1 wt % to about 20 wt % of the aqueous mixture, depending on the efficiency of vacuum removal of solvent from the preceding alkylation step shown in Scheme 1. In some embodiments, the organic solvent is present in an amount of about 5 wt % to about 10 wt % of the aqueous mixture. In some embodiments, where the intermediate being isolated is as shown in Scheme 1, the organic solvent present in the acidic aqueous mixture can vary depending on the organic solvent used in the preceding alkylation step shown in Scheme 1 or the workup procedure used in the preceding alkylation step shown in Scheme 1. In some embodiments, the organic solvent is THF or 2-Methyl-THF.

Suitable anions can be a halide, such as chloride or bromide. The buffer system used in the processes described herein is not particularly limited, and can be an aqueous solution of an alkali metal carbonate and an alkali metal bicarbonate, or an aqueous solution of an alkali metal hydroxide and an alkali metal carbonate. Suitable buffer system include, but are not limited to, an aqueous solution of sodium carbonate (Na₂CO₃) and sodium bicarbonate (NaHCO₃), an aqueous solution of potassium carbonate (K₂CO₃) and potassium bicarbonate (KHCO₃), an aqueous solution of sodium hydroxide (NaOH) and sodium carbonate (Na₂CO₃), an aqueous solution of potassium hydroxide (KOH) and potassium carbonate (K₂CO₃), an aqueous solution of monosodium phosphate (NaH₂PO₄) and disodium phosphate (Na₂HPO₄), or an aqueous solution of sodium bisulfate (NaHSO₄) and sodium sulfate (Na₂SO₄). In some embodiments, the buffer system can have a pH of from about 9 to about 12. In some embodiments, the pH of the buffer system is preferably between about 10 and about 12. In some embodiments, the pH of the buffer system is preferably between about 10 and about 11. In some embodiments, the pH of the buffer system is preferably between about 11 and about 12.

It can be advantageous to add an excess of a base, for example a carbonate base (e.g. sodium carbonate), to the buffer system. In some embodiments, the excess base can be at least 2 equivalents, about 2 equivalents to about 10 equivalents, about 2 equivalents to about 7 equivalents, about 3 equivalents to about 7 equivalents, or about 4 equivalents to about 7 equivalents of the compound of the formula (1d′-1) or the compound of formula (1d′). It has been discovered that the advantages of using excess of sodium carbonate include 1) prevent rapid degassing, i.e. the acid reacts with carbonate first to form sodium bicarbonate, and 2) sodium carbonate has significantly higher solubility in water than sodium bicarbonate salt at ambient temperature. As a result of the addition of excess carbonate, the final volume of the buffer system can be lowered, such that the ratio of buffer system to acidic aqueous mixture in the reactive crystallization is maintained at a level consistent with large scale production of pyrazole amine intermediate compound (1d). In some embodiments, the final volume ratio of the buffer system to the acidic aqueous mixture is from about 1:1 to about 10:1.

In some embodiments, the base can be an aqueous solution of an alkali metal carbonate, an alkali metal bicarbonate, an alkali metal phosphate, or ammonium hydroxide. Suitable bases include but are not limited to an aqueous solution of potassium carbonate (K₂CO₃) or an aqueous solution of sodium carbonate (Na₂CO₃). In some embodiments, the aqueous base can have a pH of from about 9 to about 12. In some embodiments, the pH of the aqueous base is preferably between about 10 and about 12. In some embodiments, the pH of the aqueous base is preferably between about 10 and about 11. In some embodiments, the pH of the aqueous base is preferably between about 11 and about 12. Depending on the base used in the process described in Scheme 2, the pH of the aqueous base solution can be maintained at a pH of about 9 by adding an aqueous solution of a strong base to the suspension mixture. Suitable examples of the strong base include 10% aqueous KOH. In some embodiments, the aqueous solution of a strong base is added via a pH pump. In some embodiments, the final volume ratio of the buffer system to the acidic aqueous mixture is from about 1:1 to about 10:1.

In some embodiments, the base can be an organic base. Suitable organic bases include but are not limited to an aqueous alkali metal acetate (such as sodium acetate), an aqueous alkali metal oxalate, a secondary alkylamine base (such as diisopropyl amine), or a tertiary alkylamine base (such as triethyl amine). In some embodiments, the final volume ratio of the buffer system to the acidic aqueous mixture is from about 1:1 to about 10:1.

It will be appreciated that the hydrolysis step shown in Scheme 1 involves the addition of a strong acid to the intermediate (1c′) to provide diacid salt (1d′). As a result of the strongly acidic conditions present during the acid hydrolysis step, the acidic aqueous mixture can have a pH of about 0 to about 2 prior to the reactive crystallization step. Because of the very low pH of the acidic aqueous mixture, the pH swing crystallization technique must be capable of adjusting the pH to a desired final pH of the suspension mixture to be in the range of from about 8 to about 10 without causing the pH to be in the range where the oiling issue of the desired pyrazole amine intermediate compound (1d) will occur (i.e. about 2.7 or about 12.5). As such, the pH of the neutralization vessel must stay within the range of about 2.7 to about 12.5. It is preferred that the pH of the neutralization vessel (containing either the buffer system or a base as described herein) will remain between about 8 and about 10.

It will be appreciated that process described herein must be amenable to the large scale production of the desired pyrazole amine intermediate compound (1d). The large scale demonstration of the standard reactive crystallization protocol (adding a base to the acid) indicated intense oiling of the product at about pH 2.7. As described above, the oiling led to processing difficulty, affected product recovery and yield, and compromised the product purity. Because the compound 3-chloro-N-ethyl-1-(pyridin-3-yl)-1H-pyrazol-4-amine (1d) is an important intermediate in the production of pesticidal thioethers, the product must meet the product production specifications. In order to meet the production specification, additional treatments such as recrystallization or reslurry of the final wet/dry cake from the hydrolysis step would be necessary with the standard approach leading to increased cycle time and further yield loss.

The reverse addition reactive crystallization technique described herein was demonstrated on large scale (50-110 g, 1 L glass reactor) as a robust crystallization approach, which completely eliminated or minimized oiling issues. This improved processability, product recovery, yield, and purity of the product without any further reprocessing of the filtered product. The feed for the reverse addition could be introduced above the surface or sub-surface, preferably sub-surface at a rate of about 2 to about 20 mL/min, preferentially at a rate of about 7 to about10 mL/min over about 30 min to about 90 min, preferably over about 30 to about 60 min to maintain the reactor temperature between about 20° C. to about 35° C.

It will be appreciated that the step of isolating can be carried out according to any method known to one of skill in the art. For example, the product can be isolated by washing the suspension mixture with deionized water. In another embodiment, the product can be isolated by on a filtration apparatus. It will be appreciated by one of skill in the art that the method for isolating is not particularly limited.

### CHEMISTRY EXAMPLES

### Materials and Methods

These examples are for illustration purposes and are not to be construed as limiting this disclosure to only the embodiments disclosed in these examples.

Starting materials, reagents, and solvents that were obtained from commercial sources were used without further purification. Melting points are uncorrected. Examples using “room temperature” were conducted in climate controlled laboratories with temperatures ranging from about 20° C. to about 24° C. Molecules are given their known names, named according to naming programs within Accelrys Draw, ChemDraw, or ACD Name Pro. If such programs are unable to name a molecule, such molecule is named using conventional naming rules. ¹⁻H NMR spectral data are in ppm (6) and were recorded at 300, 400, 500, or 600 MHz; ¹³C NMR spectral data are in ppm (6) and were recorded at 75, 100, or 150 MHz, and ¹⁹F NMR spectral data are in ppm (6) and were recorded at 376 MHz, unless otherwise stated.

### Example 1

### Hydrolysis of N-(3-chloro-1-(pyridin-3-yl)-1H-pyrazol-4-yl)-N-ethylacetamide

A light brown oil of crude N-(3-chloro-1-(pyridin-3-yl)-1H-pyrazol-4-yl)-N-ethylacetamide (about 85.43 mmol active) was dissolved in aqueous HCl (2.0 M, 169 mL, 4.0 eq.) and transferred into a 250 mL four-necked flat bottom flask leading to a dark red-orange homogeneous solution. The mixture was stirred at 80° C. for 17 h and LC (250 nm, calibrated) indicated 99.7% conversion. Reaction was stopped at 18 h and cooled down to room temperature. The dark brown solution (204.7 g) was assayed by LC analysis of a sample (424.6 mg) using di-N-propyl phthalate (180.1 mg) as an internal standard. The analysis indicated 8.12 wt %, 116.63 g product, and 90.1% in-pot yield over 2 steps.

### Example 2

### Preparation of 3-chloro-N-ethyl-1-(pyridin-3-yl)-1H-pyrazol-4-amine

A buffer system was prepared by mixing 45 mL of 0.2 M sodium carbonate with 5 mL of 0.2 M sodium bicarbonate in a reactor resulting in a pH of about 10.7 for the buffer system. 5.1 g sodium carbonate was added to 50 mL of the buffer system resulting in a pH of 11.6. 23 mL (25 g) of the hydrolyzed reaction mixture from Example 1 was loaded into a syringe and introduced to the reactor via a syringe pump at a rate of 0.383 mL/min over 1 h. The reactor temperature was maintained in a range of about 23° C. to about 25° C. After addition of the hydrolyzed mixture, the suspension mixture had a pH of about 8.47. A slight degassing was noted towards the end of the crystallization due to a lower proportion of sodium carbonate. No oiling was observed under any conditions. The resulting suspension mixture was filtered and the filtered cake was washed with about 10 g of DI water (corresponding to about 2× the mass of the wet filter cake) to yield about 4.24 g of washed wet cake. The washed wet cake was vacuum dried overnight at 50° C. to produce a 1.94 g of dry cake. The dry cake was measured to be 96.1% pure with an isolated yield of 91.9%. The yield loss to the mother liquor was about 3.1 wt % at 25° C.

### Example 3

### Preparation of 3-chloro-N-ethyl-1-(pyridin-3-yl)-1H-pyrazol-4-amine

A buffer system was prepared by mixing 45 mL of 0.2 M sodium carbonate with 50 mL of 0.2 M sodium bicarbonate in a reactor resulting in a pH of about 10.7 for the buffer system. 5.83 g sodium carbonate was added to 50 mL of the buffer system resulting in a pH of 11.6. 2.84 g of THF was added to 25.03 g of the hydrolyzed reaction mixture from Example 1. 26.35 g of the resulting 10 wt % THF containing hydrolyzed reaction mixture was loaded into a syringe and introduced to the reactor via a syringe pump at a rate of 0.383 mL/min over 1 h. The reactor temperature was maintained in a range of about 24° C. to about 28° C. After addition of the hydrolyzed reaction mixture containing 10wt % THF, the suspension mixture had a pH of about 9.09. No oiling was observed under any conditions. The resulting suspension mixture was filtered and the filtered cake was washed with about 10 g DI Water (corresponding to about 2.3× the mass of the wet filter cake) to yield 3.39 g of washed wet cake. The washed wet cake was vacuum dried overnight at 50° C. to produce 1.79 g of dry cake. The dry cake was measured to be 97.0% pure with an isolated yield of 90.6%. Yield losses to the mother liquor and wash liquor were 5.3% and 0.9% respectively at 25° C.

Table 1 is a comparison of both the approaches (standard vs reverse addition) and its impact on product purity and yield. By minimizing oiling with the reverse addition approach, the purity and the yield of pyrazole amine is maintained irrespective of the final organic solvent composition (wt % THF).

TABLE 1

| | Standard Protocol | | Reverse Addition | |
|---|---|---|---|---|
| | 0% THF | 10% THF | 0% THF | 10% THF |
| Product Purity | 96.7% | 94.1% | 96.1% | 97% |
| Isolated Yield | 94.8% | 85% | 91.9% | 90.6% |

### Example 4

### Hydrolysis of N-(3-chloro-1-(pyridin-3-yl)-1H-pyrazol-4-yl)-N-ethylacetamide

A light brown oil of crude N-(3-chloro-1-(pyridin-3-yl)-1H-pyrazol-4-yl)-N-ethylacetamide (about 0.4146 mole active) was added to aqueous HCl (4.0 M, 460 mL, 4.4 eq.) in a 1 L jacketed glass reactor leading to a dark red-orange homogeneous solution. The mixture was stirred at 90° C. for 7 h and LC (250 nm, calibrated) indicated 99.1% conversion. Reaction was stopped at 7 h and cooled down to room temperature. The dark brown solution (713.2 g) was assayed by LC analysis of a sample (540.1 mg) using di-N-propyl phthalate (144.5 mg) as an internal standard. The analysis indicated 12.9 wt %, 91.97 g product, and 99.2% in-pot yield.

### Example 5

### Preparation of 3-chloro-N-ethyl-1-(pyridin-3-yl)-1H-pyrazol-4-amine (Reverse Addition Using 5 eq. of K₂CO₃)

59 g (5 eq.) potassium carbonate was added to 150 mL of water resulting in a pH of 12.2. 149 g of the hydrolyzed reaction mixture according to Example 4 was introduced to the glass reactor via a peristaltic pump at a rate of 10.74 mL/min over 30 min. The reactor temperature was maintained in a range of about 20° C. to about 22° C. After addition of the hydrolyzed mixture, the pH of the suspension mixture was about 8. Fluffy crystals with no oiling or rinding were observed. The resulting suspension mixture was filtered and the filtered cake was washed with about 78 g of water (corresponding to about 2× the mass of the wet filter cake) to yield about 28.5 g of washed wet cake. The washed wet cake was vacuum dried overnight at 50° C. to produce a 19 g of dry cake. The dry cake was measured to be 95% pure with an isolated yield of 97%. The slurry load was 4%.

### Example 6

### Preparation of 3-chloro-N-ethyl-1-(pyridin-3-yl)-1H-pyrazol-4-amine (Reverse Addition Using 3 eq. of K₂CO₃)

57.8 g (3 eq.) potassium carbonate was added to 200 mL of water resulting in a pH of 11.9. 234.1 g of the hydrolyzed reaction mixture according to Example 4 was loaded into the glass reactor via a peristaltic pump at a rate of 10.74 mL/min over 45 min. The reactor temperature was maintained in a range of about 23° C. to about 25° C. After addition of the hydrolyzed mixture, the pH of the suspension mixture was about 9.05. Degassing was observed below pH 7.5. No oiling or rinding was observed. The resulting suspension mixture was filtered and the filtered cake was washed with about 118 g of water (corresponding to about 2× the mass of the wet filter cake) to yield about 45 g of washed wet cake. The washed wet cake was vacuum dried overnight at 50° C. to produce a 29.2 g of dry cake. The dry cake was measured to be 97-98% pure with an isolated yield of 91-93%. The slurry load was 6-7%.

### Example 7

### Preparation of 3-chloro-N-ethyl-1-(pyridin-3-yl)-1H-pyrazol-4-amine (Reverse Addition with K₂CO₃ and 10% KOH Pump.)

35.17 g (3 eq.) potassium carbonate was added to 150 mL of water resulting in a pH of 12.3. 151 g of the hydrolyzed reaction mixture according to Example 4 was loaded into the glass reactor via a peristaltic pump over 0.5 h. The reactor temperature was maintained in a range of about 24° C. to about 27° C. The pH of the buffer system was maintained at about pH 9 with the addition of 10% KOH via a pH pump. After addition of the hydrolyzed mixture, the pH of the suspension mixture was about 9.1. No degassing was observed. No oiling or rinding were observed. The resulting suspension mixture was filtered and the filtered cake was washed with about 85 g of DI water (corresponding to about 2 x the mass of the wet filter cake) to yield about 31.5 g of washed wet cake. The washed wet cake was vacuum dried overnight at 50° C. to produce 18.7 g of dry cake. The dry cake was measured to be 96.0% pure with an isolated yield of 94.2%. The slurry load was 4%.

### Example 8

### Preparation of 3-chloro-N-ethyl-1-(pyridin-3-yl)-1H-pyrazol-4-amine (Reverse Addition using ammonium hydroxide)

76 mL of 29 wt % NH₄OH was added into 100 mL of water in a 1 L glass reactor resulting in a pH of about 12. 183 mL (182 g) of the hydrolyzed reaction mixture according to Example 4 was loaded into the glass reactor via a peristaltic pump over 70 min. The reactor temperature was maintained in a range of about 20° C. to about 25° C. After addition of the hydrolyzed mixture, the pH of the suspension mixture was about 10. Larger crystal chunks without oiling or rinding were observed. The resulting suspension mixture was filtered and the filtered cake was washed with about 73 g of DI water (corresponding to about 2× the mass of the wet filter cake) to yield about 36 g of washed wet cake. The washed wet cake was vacuum dried overnight at 50° C. to produce a 23.7 g of dry cake. The dry cake was measured to be 91.4% pure with an isolated yield of 95.1%. The slurry load was 4.6%.

### Example 9

### Preparation of 3-chloro-N-ethyl-1-(pyridin-3-yl)-1H-pyrazol-4-amine (5 eq. sodium acetate)

33.6 g (5 eq.) sodium acetate was added to 100 mL of water resulting in a pH of 9.5. 141 mL (150 g) of the hydrolyzed reaction mixture according to Example 4 was loaded into the glass reactor via a peristaltic pump over 0.5 h. The reactor temperature was maintained in a range of about 20° C. to about 25° C. The pH of the buffer system was maintained at about pH 9 with the addition of 10% KOH via a pH pump. After addition of the hydrolyzed mixture, the pH of the suspension mixture was about 4.5. No degassing was observed. No oiling or rinding was observed. The resulting suspension mixture was filtered and the filtered cake was washed with about 92.3 g of DI water (corresponding to about 2 x the mass of the wet filter cake) to yield about 43.9 g of washed wet cake. The washed wet cake was vacuum dried overnight at 50° C. to produce a 17.9 g of dry cake. The dry cake was measured to be 90.8% pure with an isolated yield of 86.5%. The slurry load was 5.7%.

### Example 10

### Preparation of 3-chloro-N-ethyl-1-(pyridin-3-yl)-1H-pyrazol-4-amine (8.5 eq. sodium acetate)

58.6 g (8.4 eq.) sodium acetate was added to 150 mL of water resulting in a pH of 9. 160 mL (150.0 g) of the hydrolyzed reaction mixture according to Example 4 was loaded into the glass reactor via a peristaltic pump over 1.5 h. The reactor temperature was maintained in a range of about 24° C. to about 26° C. The pH of the buffer system was maintained at about pH 9 with the addition of 10% KOH via a pH pump. After addition of the hydrolyzed mixture, the pH of the suspension mixture was about 4.4. No degassing was observed. No oiling or rinding were observed. The resulting suspension mixture was filtered and the filtered cake was washed with about 94.2 g of DI water (corresponding to about 2 x the mass of the wet filter cake) to yield about 27.2 g of washed wet cake. The washed wet cake was vacuum dried overnight at 50° C. to produce a 18.4 g of dry cake. The dry cake was measured to be 96.7% pure with an isolated yield of 94.9%. The slurry load was 5%.

### Example 11

### Preparation of 3-chloro-N-ethyl-1-(pyridin-3-yl)-1H-pyrazol-4-amine (5 eq. triethylamine, TEA)

9.55 g (5 eq.) triethylamine was added to 30 mL DI water resulting in a pH of 11.2. 40.09 g of the hydrolyzed reaction mixture according to Example 4 was loaded into a syringe and introduced to the glass reactor via a syringe pump at a rate of 0.57 mL/min over 1 h. The reactor temperature was maintained in a range of about 25° C. to about 33° C. After addition of the hydrolyzed mixture, the pH of the suspension mixture was about 2.96. No oiling was observed. The resulting suspension mixture was filtered and the filtered cake was washed with about 20 g of DI water (corresponding to about 2× the mass of the wet filter cake) to yield about 6 g of washed wet cake. The washed wet cake was vacuum dried overnight at 50° C. to produce a 4 g of dry cake. The dry cake was measured to be 95.5% pure with an isolated yield of 82.7%. The yield loss to the mother liquor was about 17.3 wt % at 25° C.

### COMPARATIVE EXAMPLES

### Example CE-1 Preparation of 3-chloro-N-ethyl-1-(pyridin-3-yl)-1H-pyrazol-4-amine

Example CE-1 is a comparative example wherein 40.06. g of the hydrolyzed reaction mixture of Example 1 of 8.12 wt % purity was loaded in the reactor. 11.61 g of 25 wt % aqueous NaOH solution was introduced to the reactor via a syringe pump over an hour at a rate of 0.15 mL/min. The reactor temperature was maintained in a range of about 20° C. to about 30° C. throughout the length of addition. A distinct oiling was observed on the reactor surface at about pH 2.7 indicating that oiling is intrinsic to the molecule and the system. After addition of the caustic solution, the resulting suspension was filtered and the filter cake was washed with about 2×8 mL of DI water. The washed wet cake was dried overnight in a vacuum oven at 50° C. to produce 3.19 g of yellowish brown dry cake. The dry cake was measured to be 96.7 wt % pure with an isolated yield of 94.8% (determined by quantitative LC assay).

### Example CE-2

### Preparation of 3-chloro-N-ethyl-1-(pyridin-3-yl)-1H-pyrazol-4-amine

Example CE-2 is a comparative example wherein 40 g of the hydrolyzed reaction mixture of Example 1 having 8.12 wt % purity was mixed with 4.44 g THF. The resulting mixture containing 10 wt % THF was loaded in the reactor. 11.68 g of 25 wt % aqueous NaOH solution was introduced to the reactor via a syringe pump over an hour at a rate of 0.15 mL/min. The reactor temperature was maintained within a range of about 20° C. to about 30° C. throughout the length of addition. As observed in the 1 L scale, a distinct oiling was noticed on the reactor surface at about pH 2.8. Just before oiling started, the reactor mass turned cloudy indicating the presence of a second liquid phase. Solids started precipitating out at about pH 2.5. At the completion of the 25 wt % aqueous NaOH addition, the end point pH was about 10.7. With continued addition, the oil gradually started reacting away and solids started forming on the reactor surface. This observation was very similar to that in larger 1 L scale. The end-point pH was about 8.5. The resulting suspension mixture was filtered and the filtered cake was washed with about 3×10 mL DI water to yield about 6.75 g of wet washed cake. The wet washed cake was vacuum dried overnight at 50° C. to produce about 2.97 g of dark brown dry cake. LC analysis indicated a 94.1 wt % purity with about 85% yield. Illustratively, for this example, the entrapped oil resulted in dark brown colored lower purity product. Yield loss to the mother liquor was 6.5% (as expected due to the higher solubility of 3-chloro-N-ethyl-1-(pyridin-3-yl)-1H-pyrazol-4-amine amine in THF).

### Example CE-3

### Preparation of 3-chloro-N-ethyl-1-(pyridin-3-yl)-1H-pyrazol-4-amine (Standard Addition Using NH₄OH)

Example CE-3 is a comparative example wherein 182.1 g of the hydrolyzed reaction mixture according to Example 1 was loaded into the reactor. 66.2 g of 29 wt % aqueous NH₄OH solution was introduced to the reactor via a peristaltic pump over 30 min. The reactor temperature was maintained within a range of about 18° C. to about 32° C. throughout the length of addition. A distinct oiling was observed on the reactor surface at about pH 3 and about pH 8.5. After addition of the NH₄OH solution, the resulting suspension mixture was filtered and the filter cake was washed with about 100 mL of water. The washed wet cake was dried overnight in a vacuum oven at 50° C. to produce 53.67 g of yellowish brown dry cake. The dry cake was measured to be 92.1 wt % pure with an isolated yield of 88.9% (determined by quantitative LC assay).

### Example CE-4

### Preparation of 3-chloro-N-ethyl-1-(pyridin-3-yl)-1H-pyrazol-4-amine (Con Addition of Acid and Base)

A buffer system was prepared by mixing 0.09 g of solid sodium hydroxide with 0.87 g of solid sodium bicarbonate to 400 mL of DI water in a 1 L glass reactor. 226.2 g of the hydrolyzed reaction mixture according to Example 4 was loaded into the glass reactor via a peristaltic pump at a rate of 7 mL/min over 1 h. 248.4 g of 10 wt % sodium hydroxide solution was continuously-added via a pH metering pump while maintaining the pH to about 9.5 during the addition. The reactor temperature was maintained between 23° C. and 27° C. After addition of the hydrolyzed mixture, the pH of the suspension mixture was about 9.5. Oiling and rinding were observed. The resulting suspension mixture was filtered and the filtered cake was washed with about 95 g of DI water (corresponding to about 2.5× the mass of the wet filter cake) to yield about 32.03 g of washed wet cake. The washed wet cake was vacuum dried overnight at 50° C. to produce a 19.7 g of dry cake. The dry cake was measured to be 95% pure with an isolated yield of 68%. The slurry load was 2.5%.

### Example CE-5

### Preparation of 3-chloro-N-ethyl-1-(pyridin-3-yl)-1H-pyrazol-4-amine (Con Addition of Acid and Base)

A buffer system was prepared by mixing 0.27 g of 45 wt % potassium hydroxide solution with 1.05 g of solid potassium bicarbonate to 400 mL DI water in a 1 L glass reactor. 218 g of the hydrolyzed reaction mixture according to Example 4 was loaded into the glass reactor via a peristaltic pump at a rate of 7 mL/min over 40 min. 765.4 g of 10 wt % potassium hydroxide solution was continuously-added via a pH metering pump maintaining the pH to about 9.5 during the addition. The reactor temperature was maintained in a range of about 24° C. to about 26° C. After addition of the hydrolyzed mixture, the pH of the suspension mixture was about 9.9. Oiling and rinding were observed. The resulting suspension mixture was filtered and the filtered cake was washed with about 64 g of water (corresponding to about 2× the mass of the wet filter cake) to yield about 21.52 g of washed wet cake. The washed wet cake was vacuum dried overnight at 50° C. to produce a 11.8 g of dry cake. The dry cake was measured to be 94% pure with an isolated yield of 44%. The slurry load was 1%.

### Example CE-6

### Preparation of 3-chloro-N-ethyl-1-(pyridin-3-yl)-1H-pyrazol-4-amine (Con Addition of Acid and Base to Water)

100 mL DI water was pre-loaded into a 1 L glass reactor. 150.3 g of the hydrolyzed reaction mixture according to Example 4 was loaded into the glass reactor via a peristaltic pump at a rate of 7 mL/min over 30 min. 316.6 g of 10 wt % potassium hydroxide solution was continuously-added via a pH metering pump maintaining the pH to about 9.5 during the addition. The reactor temperature was maintained in a range of about 24° C. to about 28° C. After addition of the hydrolyzed mixture, the pH of the suspension mixture was about 9.9. Oiling and rinding were observed. The resulting suspension mixture was filtered and the filtered cake was washed with about 64 g of water (corresponding to about 2.5× the mass of the wet filter cake) to yield about 23.2 g of washed wet cake. The washed wet cake was vacuum dried overnight at 50° C. to produce a 10.9 g of dry cake. The dry cake was measured to be 94% pure with an isolated yield of 58.2%. The slurry load was 1.7%.

What is claimed is:

1. A process for producing a compound of the formula (1d-1) 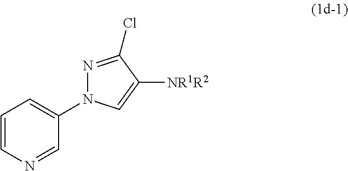
in purified form, wherein R¹ and R² are each independently H, C₁-C₄ alkyl, C₁-C₄ alkynyl, or —C(O)C₁-C₄ alkyl, comprising
a. contacting a compound of the formula (1d′-1) 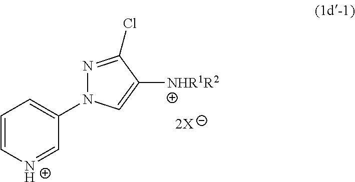
wherein R¹and R² are each independently H, C₁-C₄ alkyl, C₁-C₄ alkynyl, or —C(O)C₁-C₄ alkyl, X⁻ is an anion, with a base or a buffer system at a pH of about 7 to about 12 and at a temperature of from about 20° C. to about 35° C., to provide a suspension mixture of the compound of the formula (1d-1) as a solid product suspended in the base or the buffer system.

2. The process of claim 1, wherein R¹ is H and R² is ethyl.

3. The process of claim 1 or 2, further comprising
b. isolating the compound of the formula (1d-1) to provide the compound of the formula (1d-1) in purified form.

4. The process of claim 1 or 2, further comprising
c. drying the compound of the formula (1d-1) in purified form in vacuo.

5. The process of claim 1 or 2, wherein the step of contacting comprises adding an acidic aqueous mixture comprising the compound of the formula (1d′-1) to the base or the buffer system.

6. The process of claim 5, wherein the acidic aqueous mixture comprises at least one organic solvent.

7. The process of claim 6, wherein the organic solvent is present in an amount of about 0.1 wt % to about 20 wt % of the acidic aqueous mixture or the organic solvent is present in an amount of about 5 wt % to about 10 wt % of the acidic aqueous mixture.

8. The process of claim 6, wherein the organic solvent is THF or 2-Me-THF.

9. The process of claim 5, wherein the acidic aqueous mixture has a pH of about 0 to about 2.

10. The process of claim 5, wherein the final volume ratio of the base or the buffer system to the acidic aqueous mixture is from about 1:1 to about 10:1.

11. The process of claim 1 or 2, wherein the buffer system is an aqueous solution of an alkali metal carbonate and an alkali metal bicarbonate, or an alkali metal hydroxide and an alkali metal carbonate.

12. The process of claim 11, wherein the buffer system is an aqueous solution of sodium carbonate (Na₂CO₃) and sodium bicarbonate (NaHCO₃), an aqueous solution of potassium carbonate (K₂CO₃) and potassium bicarbonate (KHCO₃), an aqueous solution of sodium hydroxide (NaOH) and sodium carbonate (Na₂CO₃), an aqueous solution of potassium hydroxide (KOH) and potassium carbonate (K₂CO₃), an aqueous solution of monosodium phosphate (NaH₂PO₄) and disodium phosphate (Na₂HPO₄), or an aqueous solution of sodium bisulfate (NaHSO₄) and sodium sulfate (Na₂SO₄).

13. The process of claim 1 or 2, wherein the buffer system has a pH of from about 9 to about 12.

14. The process of claim 1 or 2, wherein the base is an aqueous solution of an alkali metal carbonate, an alkali metal bicarbonate, an alkali metal phosphate, or ammonium hydroxide.

15. The process of claim 1 or 2, wherein the base is an aqueous solution of potassium carbonate (K₂CO₃) or sodium carbonate (Na₂CO₃).

16. The process of claim 15, wherein the step of contacting is maintained at a pH of about 9 by adding an aqueous solution of a strong base to the suspension mixture.

17. The process of claim 16, wherein the aqueous solution of a strong base is about 10% aqueous KOH or NaOH.

18. The process of claim 1 or 2, wherein the base is an organic base.

19. The process of claim 18, wherein the organic base is an aqueous alkali metal acetate, an aqueous alkali metal oxalate, a secondary alkylamine base or a tertiary alkylamine base.

20. The process of claim 19, wherein the alkali metal acetate is sodium acetate or potassium acetate.

21. The process of claim 19, wherein the tertiary alkylamine base is triethyl amine.

22. The process of claim 1 or 2, wherein the final pH of the suspension mixture is from about 8 to about 10.

23. The process of claim 1 or 2, wherein an excess of a base is added to the buffer system.

24. The process of claim 23, wherein the excess base can be about 2 equivalents to about 10 equivalents relative to the compound of formula 1d′-1.

25. The process of claim 23, wherein the excess base is potassium carbonate (K₂CO₃) or sodium carbonate (Na₂CO₃).
